(19) United States
(12) Patent Application Publication
(10) Pub. No.: US 2003/0224470 A1
(43) Pub. Date: Dec. 4, 2003

(54) Method of diagnosing statin myopathy

(76) Inventor: Paul S Phillips, Coronado, CA (US)

Correspondence Address: BROWN, MARTIN, HALLER & MCCLAIN LLP, 1660 UNION STREET, SAN DIEGO, CA, 92101-2926, US

(21) Appl. No.: 10/388,032

(22) Filed: Mar. 12, 2003

Related U.S. Application Data
(60) Provisional application No. 60/363,435, filed on Mar. 12, 2002.

(51) Int. Cl. G01N 33/53; G01N 33/537; G01N 33/543; G01N 33/00

(52) U.S. Cl. 435/7.92; 435/7.1; 436/129

(57) ABSTRACT

The use of statins for the control of hypercholesterolemia is continually increasing. Although the side effects of statins are relatively uncommon, muscle myopathies may develop in response to statin therapy. The invention is the discovery of a correlation between statin related myopathy based on the presence of 3-methylglutaconic acid (3MGA) in the urine. This correlation allows for the screening of both symptomatic and asymptomatic individuals for statin related myopathy.

### CROSS-REFERENCES TO RELATED APPLICATIONS

This application claims the benefit of priority of United States provisional application Serial No. 60/363,435 filed Mar. 12, 2002 which is incorporated herein by reference in its entirety.

### BACKGROUND OF THE INVENTION

3-Hydroxy-3-methylglutaryl coenzyme A (HMG-CoA) reductase inhibitors (statins) are the mainstay of therapy for hypercholesterolemia because of their effectiveness and exceptional safety profile. Nonetheless, many patients treated with statins have muscle symptoms, and some patients develop severe muscle toxicity. Little is known about the mechanism by which statin therapy leads to muscle toxicity. The recent withdrawal of cerivastatin from the U.S. market has highlighted both our ignorance and the need for postmarketing surveillance of these therapies (Farmer Lancet 358:1383-5; 2001).

There are credible reports of patients who have muscle symptoms and normal serum creatine kinase levels while receiving statins. However, there is no way to determine if the source of the myopathy is the statin or a non-related cause. As statin therapy becomes used more aggressively in larger groups of patients, the importance of even minor toxic side effects of this therapy is magnified. Early detection of patients who may suffer toxic muscular reactions to these drugs would be extremely useful.

### SUMMARY OF THE INVENTION

The invention is the discovery that an elevated level of 3-methylglutaconic acid (3MGA) in the urine is strongly correlated with statin associated myopathy confirmed by muscle biopsy and functional studies to determine muscle weakness. In all individuals with elevated levels of 3MGA in the urine, termination of statin therapy resulted in resolution of myopathy with no additional intervention. Re-administration of statin resulted in myopathy. These results demonstrate a direct relationship between statin therapy, elevated 3MGA in the urine and myopathy.

The invention is a diagnostic test to identify individuals with myopathy that may be a result of statin therapy by detection of 3MGA in the urine. The level of 3MGA detected in individuals with statin myopathy in urine organic acid analysis by spectroscopic techniques is between about 9 and 100 millimole/mole creatine, with a typical range observed between 9 and 50 millimole/mole creatine. 3MGA levels up to about 200 millimole/mole creatine can be indicative of statin related myopathy. Although these ranges are higher than found in the general population, levels up to 50 millmole/mole creatine would not be considered abnormal. Disorders diagnosed based on increased levels of 3MGA in the urine are typically pediatric disorders in which 3MGA levels are found to be substantially in excess of 100 millimole/mole creatine (e.g. Barth syndrome, an X-linked cardiomyopathy with neutropenia and Costeff syndrome, a slowly progressive neurodegenerative disorder).

The invention is a screening method to identify individuals who may develop statin myopathy before symptoms are present. After about 6 weeks of statin therapy, a test is performed to detect 3MGA in the urine. If an elevated level of 3MGA is detected, the individual is identified as being susceptible to develop statin related myopathy and is typically taken off statin therapy and other methods to control hypercholesterolemia are used. This regimen is preferably combined with a baseline measurement of 3MGA either before the initiation of statin therapy or after a washout period of at least two weeks after statin therapy is terminated. This is to insure that the increased 3MGA level is related to the statin therapy and not some other cause.

### BRIEF DESCRIPTION OF THE TABLE

TABLE 1 is a summary of the lab values obtained for the four patients enrolled in the study.

### DETAILED DESCRIPTION AND PREFERRED EMBODIMENTS

Randomized trials and community surveillance have demonstrated an extremely low incidence of serious muscle toxicity in patients receiving statins: 1 case per 10,000. Despite this low incidence, many patients attribute their muscle symptoms to statin therapy. The invention is the discovery that patients with statin myopathy who have normal creatine levels have an increased level of 3MGA in the urine. Increased levels of 3MGA in the urine were strongly correlated with abnormalities in muscle biopsy, functional capacity and decreased muscle strength. Upon withdrawal of statin therapy, the myopathies were found to resolve without additional interventions.

This discovery allows for the diagnosis of statin related myopathies non-invasively based on an elevated level of 3MGA in the urine rather than muscle biopsy which is invasive and painful. Moreover, a muscle biopsy can reveal damage, but does not necessarily indicate the source of the myopathy. This discovery allows for the screening for individuals who will develop statin related myopathies before the development of symptoms by detection of elevated levels of 3MGA in the urine preventing damage to the muscle.

The invention is not limited by the method of detection of 3MGA in the urine. Presently, 3MGA is typically detected using gas chromatography mass spectrometry (GCMS) or tandem mass spectrometry techniques. The development of tests for the detection of organic molecules in bodily fluids is well within the ability of those skilled in the art. Any quantitative or qualitative method able to detect 3MGA with the sensitivity required can be used to practice the method of the invention.

An elevated level of 3MGA is defined as a baseline value plus two standard deviations as determined by individual testing laboratories. The baseline value may be compared to a control sample for an individual while the individual is not undergoing statin therapy, either before the initiation of statin therapy or after an appropriate washout period. Alternatively, baseline may be defined by the laboratory as an average normal reading. The standard deviations are determined from this number and are added to either baseline number.

As some variance may exist between testing laboratories, exact values indicative of myopathy may vary. All of the values presented herein were obtained at the University of California San Diego clinical testing laboratory. In that laboratory, two standard deviations from the average background level is 9 millimoles/mole creatine. Based on values obtained in that laboratory, values indicative of statin related myopathy are between about 9 and 200 millimoles/mole creatine, typically between about 9 to 100 millimoles/mole creatine, most typically between about 9 and 50 millimoles/mole creatine. Levels of 3MGA are normalized to the amount of creatine present in the sample to account for the hydration level of the patient. Although these levels are higher than average in the general population, values from 9 to 100 millimoles/mole creatine would not be considered to be in the abnormal range.

Quantitative, semi-quantitative and qualitative tests for the presence of specific organic compounds in urine or other bodily fluids are well known. Such tests include enzyme linked immunosorbent assay (ELISA), radioimmune assay (RIA), bead agglutination test and other antibody based tests, as well as chemical based tests. Initial screening for the presence of 3MGA in the urine can be done using a qualitative bead agglutination or chemical based home-test, similar to a home pregnancy test. Individuals with a positive result can be subjected to a more quantitative test by a physician or other individual skilled in the art. The method of detection of 3MGA in the urine is a matter of choice that can be readily made by one skilled in the art.

The association of increased levels of 3MGA with statin myopathy was discovered in a study of four patients identified among the first 20 patients randomly assigned in an ongoing, unfunded, doubleblinded, crossover clinical trial that had been approved by an investigational review board. Patients who had muscle symptoms during statin therapy that resolved when they were not receiving statins for 2 weeks were recruited from a clinical research group. Eligible patients had to have normal creatine kinase levels while they were experiencing symptoms. End points were 1) whether patients could accurately identify blinded statin therapy, 2) standard measures of functional capacity and muscle strength, 3) standard measures of biochemical properties of blood, urine and muscle specimens and 4) analysis of muscle biopsy for pathology. The muscular strength tests during both placebo use and statin therapy included hip abduction and flexion measured by Nicholas Manual Muscle Tester (Lafayette Instrument Co., Lafayette, Ind.) The standard measures for biochemical alterations included blood, urine and muscle biopsy analysis for levels of endogenous factors and drug. Analysis of muscle biopsy for pathology included general histology of the samples and detection of various markers involved in metabolism.

### Patient 1

Patient 1 developed muscle aches and decreased exercise tolerance during 4 years of simvastatin therapy. She found it difficult to ascend one flight of stairs without resting to relieve severe leg aching. Symptoms did not change during a brief trial of atorvastatin. After the patient entered the trial, her muscle symptoms markedly resolved during the 2-week washout period and the blinded placebo phase. Muscle aching recurred within 48 hours of initiation of the statin phase. After 2 weeks of statin therapy, the patient again found it difficult to ascend a flight of stairs because of leg pain and weakness. She repeated the protocol and again experienced symptoms while receiving statin; the symptoms again resolved during the placebo phase. Results of serum and urine biochemical analyses are reported in TABLE 1. Termination of statin therapy resulted in quantitatively increased muscle strength and decreased 3MGA levels in the urine.

Muscle biopsy revealed extensive lipid-filled vacuoles distributed within the myofibers, along with cytochrome oxidase-negative myofibers. The patient's muscle symptoms and hip weakness improved 3 months after she had discontinued statin therapy. At that point, repeated muscle biopsy showed complete resolution of the pathologic abnormalities.

### Patient 2

Patient 2 developed muscle pains and weakness while receiving lovastatin, 40 mg/d. She could not open jars or snap her fingers. Her physician changed her therapy to niacin for 2 years. Each time simvastatin was added to improve her lipid control, the patient developed muscle aches, weakness, and shortness of breath with exertion. She entered the double-blinded trial for two separate evaluations and correctly identified the statin phases because of reproduction of her symptoms. The patient had muscle weakness and normal creatine kinase levels during each statin phase (TABLE 1). Termination of statin therapy resulted in quantitatively increased muscle strength and decreased 3MGA levels in the urine.

Muscle biopsy revealed accentuated lipid droplet accumulation and cytochrome oxidase-negative muscle fibers that were consistent with myopathy. Repeated muscle biopsy performed 3 months after discontinuation of statin therapy revealed complete resolution of the abnormal lipid stores.

### Patient 3

Patient 3 developed aching and weakness of the shoulder, hip, and thigh while taking atorvastatin, 20 mg/d, for 2 years. During a 2-week vacation, he forgot his medication and noticed a substantial resolution of his muscle symptoms. He entered the trial for two separate evaluations and correctly identified the statin arm both times after developing weakness; his creatine kinase level was normal at both evaluations (TABLE 1). Termination of statin therapy resulted in quantitatively increased muscle strength and decreased 3MGA levels in the urine.

A muscle biopsy performed during atorvastatin therapy revealed increased lipid droplets, ragged red fibers, and cytochrome oxidase—negative muscle fibers, all consistent with lipid myopathy. Repeated biopsy done after the patient had not received statin therapy for 5.5 months showed resolution of the increased lipid droplets.

### Patient 4

Patient 4 began receiving atorvastatin, 10 mg/d, for combined hyperlipidemia, and within 2 weeks he noticed leg aches when climbing stairs. His symptoms resolved 2 weeks after he had stopped taking atorvastatin. Upon enrollment in the trial, he correctly identified the blinded statin therapy and had measurable hip weakness despite a normal creatine kinase level (TABLE 1). Termination of statin therapy resulted in quantitatively increased muscle strength.

His muscle biopsy demonstrated cytochrome oxidase-negative fibers. Electron microscopy showed abnormal lipid droplets surrounded by electron-dense membranous debris. The patient did not undergo biopsy after discontinuation of statin therapy.

### Testing

The similar clinical, pathologic, and biochemical features of the four patients suggest that the patients have the same type of myopathy. More important, these features are convincingly related to statin therapy and fulfill the accepted criteria for an adverse drug reaction: 1) Muscle symptoms occurred when the patients were receiving statins in a double-blinded fashion, 2) these symptoms normalized when the patients received placebo, 3) muscle toxicity recurred upon re-exposure to the statin, as it had occurred upon previous exposures, 4) the adverse reaction was confirmed by pathologic and biochemical findings, and 5) the pathologic abnormality reversed upon discontinuation of statin therapy.

To further confirm that the myopathies were statin based, a series of biochemical tests was performed to exclude possible causes of myopathy other than statin therapy. Testing did not reveal any evidence of carnitine deficiency or hypothyroidism in these patients.

The histochemical findings in these four patients were surprisingly similar. Unusually prominent accumulations of lipid were present in the type 1 fibers on oil red O stains or electron microscopy in all four patients. Other evidence of myopathy included cytochrome oxidase-negative staining fibers and ragged red fibers. These myopathic findings reversed when three of these patients had repeated biopsy while not receiving statins. Increased lipid stores, cytochrome oxidase-negative myofibers, and ragged red fibers are features of mitochondrial respiratory chain dysfunction and suggest myopathy due to a metabolic abnormality. Although some of these findings may rarely occur in myofibers because of aging, the pattern of increased lipid is unusual. It has previously been described as a drug reaction and as a consequence of coenzyme Q10 deficiency.

Increased levels of 3MGA in the urine was found in the three patients tested in this study while they were undergoing statin therapy. 3MGA was absent when they were not receiving statins. Five additional patients with biopsy confirmed myopathy were tested for increased levels of 3MGA in the urine. Four were found to have increased 3MGA levels. A series of six other patients who did not have muscle symptoms while receiving long-term statin therapy were tested, and found to have no abnormal organic acidurias in them. Although the sample size is small, the predictive value of the presence of 3MGA in the urine within the sample is high (87.5% sensitive and 100% specific).

All four patients in the study had normal statin levels (TABLE 1). Thus, these patients did not become myopathic because of too much drug or inadequate clearance. However, the development of toxicity at normal statin activity levels suggests that the patients might have an underlying metabolic vulnerability.

The creatine kinase level remained normal in these patients every time they became myopathic both before and during the trial. The presence of a normal creatine kinase level has previously been used to argue against myopathy in patients with muscle symptoms during statin therapy. These cases demonstrate that serum creatine kinase level is an inadequate test for statin-associated myopathy.

### Statin Related Myopathy in the General Population

The study above demonstrated the reversibility of statin related myopathy in the patients studied. A statin-related myopathy web site was established to determine if the reversibility of the statin based myopathy observed in the small sample could be observed in a larger population. During the course of the study there were over 18,000 visits to the site with 614 individuals completing myopathy surveys. Of the 614 respondents, 35% stated that their physician believed that the myopathy was statin related, and 50% stated that their symptoms improved upon termination of statin therapy. There was a large overlap in the two groups. These data demonstrate that statin based myopathy is widespread and that proper diagnosis can result in amelioration of symptoms for a large number of individuals.

### EXAMPLE 1

Evaluation of a patient undergoing statin therapy with muscle weakness and soreness. A patient complains to his physician of increasing muscle pain and weakness after having undergone statin therapy for at least two months. A urine sample is obtained by the physician and sent to the laboratory for analysis of urine organic acids. The analysis reveals that 3MGA levels are 22 mmole/mole of creatine. The physician terminates statin therapy with the patient and introduces other methods to reduce cholesterol (e.g. better diet, exercise, non-statin pharmacological interventions). The patient is monitored at regular intervals during the washout period when the drug is cleared from the body (e.g. monthly, every two months) until muscle strength returns and pain abates. The patient is again tested for 3MGA levels which have returned to baseline, essentially zero.

### EXAMPLE 2

Evaluation of a patient at the initiation of statin therapy. A physician determines that the cholesterol of a patient is sufficiently high to warrant pharmacological interventions. Before initiating statin therapy, a urine sample is collected from the patient and analyzed for levels of 3MGA. No 3MGA is found in the urine. After 6 weeks of statin therapy, the patient uses a qualitative home test to detect 3MGA in the urine, typically an apparatus that can be held in the urine stream to detect 3MGA, similar to a home pregnancy test. After a specified time period, the test apparatus is observed for a color change.

If a color change is observed, the patient contacts the physician for further tests and/or analysis. Statin therapy is typically stopped and alternative therapies are considered.

If no color change is observed, the patient continues with statin therapy as originally instructed by the physician.

### EXAMPLE 3

Quantitative testing methods. Due to the limited demand for testing for 3MGA in urine, no test exists specifically for its detection. Typically 3MGA is detected during a complete urine organic acid analysis which is performed using tandem mass spectrophotometry or gas chromatography mass spectrophotometry. Such methods are routine and well known to those skilled in the art. Such analyses are available through many clinical laboratories at major hospitals.

An ELISA assay can be readily developed to detect the presence of 3MGA in the urine. ELISA assays are ideal for the analysis of large numbers of samples as they can be readily performed by machine. Antibodies, preferably monoclonal antibodies, can be produced to 3MGA by methods well known to those skilled in the art for use in ELISA assays. As with spectrophotometric analysis, the amount of 3MGA present can be normalized the to amount of creatine in the urine. Such methods are well known to those skilled in the art.

### EXAMPLE 4

Qualitative testing methods. Qualitative tests are ideal for home use and screening by patients as they are relatively inexpensive and easy to use. One such test is a bead agglutination assays, similar to a home pregnancy test. Such tests are well known to those skilled in the art. The test apparatus includes an absorbent end to be held in the urine stream to collect the sample. The absorbent end is functionally connected to a reaction chamber to allow a portion of the urine sample to be transferred to the reaction chamber. Colored beads coated with antibody to 3MGA are dispersed throughout the reaction chamber. If 3MGA is present, the dispersed beads aggregate making the color visible. If no 3MGA is present, the beads do not aggregate and no color change is visible. Similar tests can be developed based on chemical reactions with 3MGA resulting in a color change, or not, depending on the presence or absence of 3MGA respectively.

Although an exemplary embodiment of the invention has been described above by way of example only, it will be understood by those skilled in the field that modifications may be made to the disclosed embodiment without departing from the scope of the invention, which is defined by the appended claims.

TABLE 1

| | | | | | | | | | Creative | | |
|---|---|---|---|---|---|---|---|---|---|---|---|
| | | | | | | | | | Kinase | | |
| | | | | | | | | | Level | | |
| | | | | Hip Abduction | | | | | during | 3-Methylglutaconic | |
| | | | | (Strength) | | Hip Flexion (Strength) | | Statin | Statin | acid levels [symbol] | |
| | Age | Lipid Levels | Statin Dosage | On Statin | Off Statin | On Statin | Off Statin | Level | Therapy‡ | On Statin | Off Statin |
| PATIENT | y | mmol/L | mg/d | kg | kg | kg | kg | ngEq/cm³ | μkat/L | mmol/mol | mmol/mol |
| 1 | 76 | *T.C. 7.63 | Cerivistatin, 0.4 | 4.1 | 7.8 | 8.0 | 16.8 | 1.4 (Sim- | 0.63 | 21 | 0 |
| | | Tg 4.22 | Pravastatin, 40 | | | | | vastatin) \| | | | |
| | | LDLC 4.71 | Simvastatin, 80 | | | | | | | | |
| | | HDLC 0.98 | Atorvastatin, 20 | | | | | | | | |
| 2 | 66 | T.C. 9.59 | Simvastatin, 40 | 11.6 | 12.9 | 9.6 | 10.1 | 16.3 (Sim- | 0.73 | 11 | 0 |
| | | Tg 3.87 | Lovastatin, 40 | | | | | vastatin) \| | | | |
| | | LDLC 6.47 | | | | | | | | | |
| | | HDLC 1.34 | | | | | | | | | |
| 3 | 66 | T.C. 5.95 | Atorvastatin, 20 | 6.3 | 9.0 | 14.3 | 16.3 | 10.9 (Ator- | 2.30 | ND | ND |
| | | Tg 2.26 | | | | | | vastatin) [symbol] | | | |
| | | LDLC 3.39 | | | | | | | | | |
| | | HDLC 1.53 | | | | | | | | | |
| 4 | 62 | T.C. 5.87 | Atorvastatin, 10 | 12.9 | 14.8 | 24.1 | 27.2 | NA | 1.39 | 16 | 3 |
| | | Tg 2.37 | | | | | | | | | |
| | | LDLC 3.90 | | | | | | | | | |
| | | HDLC 0.88 | | | | | | | | | |
| *TC = Total Cholesterol; Tg = Triglycerides; HDLC = high-density lipoprotein cholesterol; LDLC = low-density lipoprotein cholesterol; NA = not available; ND = not done. To convert lipid values to mg/dL, divide by 0.0259. | | | | | | | | | | | |
| †Statin at time of biopsy is listed first, followed by all other statins causing symptoms for that patient. No patient was taking macrolide antibiotics, cyclosporine, fluconazole, or fibrates or was consuming large amounts of grapefruit juice during the study. Thyroid-stimulating hormone levels in both patients with history of L-thyroxine use were normal. No patient was consuming large amounts of alcohol. Electromyography, nerve conduction studies, and | | | | | | | | | | | |
| #magnetic resonance imaging findings were normal in patients 2 and 3. | | | | | | | | | | | |

‡Normal range, 0-3.26 μkat/L; [symbol] Normal range, 0-9 mmol/mol creatinine; | Simvastatin level within therapeutic range; [symbol] Atorvastatin level within therapeutic range.

I claim:

1. A method of identifying statin related myopathy in an individual comprising:
collecting a urine sample from the individual undergoing statin therapy; and
analyzing the sample for 3-methylglutaconic acid (3MGA) wherein an elevated level of 3MGA in the sample is correlated with statin related myopathy.

2. The method as in claim 1, wherein the elevated level of 3MGA comprises at least a baseline value plus two standard deviations of the average normal reading.

3. The method as in claim 1, wherein the elevated level is at least about 9 millimole/mole creatine.

4. The method as in claim 1, wherein the elevated level of 3MGA is between about 9 and 200 millimole/mole creatine.

5. The method as in claim 1, wherein the elevated level of 3MGA is between about 9 and 100 millimole/mole creatine.

6. The method as in claim 1, wherein the elevated level of 3MGA is between about 9 and 50 millimole/mole creatine.

7. The method as in claim 1, wherein the elevated level is an increased concentration of 3MGA in the urine as compared to the concentration of 3MGA in the urine in the same individual while not taking statins.

8. The method as in claim 1, wherein the concentration of 3MGA in the urine of the individual is tested before starting statin therapy.

9. The method as in claim 1, wherein the concentration of 3MGA in the urine of the individual is tested after a washout period after termination of statin therapy.

10. The method as in claim 1, wherein the individual has symptoms of statin related myopathy.

11. The method as in claim 1, wherein the individual does not have symptoms of statin related myopathy.

12. The method as in claim 1, wherein the concentration of 3MGA in the urine of the individual is tested before starting statin therapy.

13. The method as in claim 1, wherein the concentration of 3MGA in the urine of the individual is tested after a washout period after termination of statin therapy.

14. The method as in claim 1, wherein the analysis is a quantitative analysis.

15. The method as in claim 14, wherein the quantitative analysis is performed using spectrophotometric methods.

16. The method as in claim 14, wherein the quantitative analysis is performed using an enzyme linked immunosorbent assay (ELISA).

17. The method as in claim 14, wherein the quantitative analysis is performed using a radioimmune assay (RIA).

18. The method as in claim 1, wherein the analysis is a qualitative analysis.

19. The method as in claim 18, wherein the qualitative analysis is performed using a bead agglutination assay.

20. The method as in claim 8, wherein the qualitative analysis is performed using a chemical bases analysis.
